(19) United States
(12) Patent Application Publication
(10) Pub. No.: US 2004/0127395 A1
(43) Pub. Date: Jul. 1, 2004

(54) Use of histamine H4 receptor modulators for the treatment of allergy and asthma

(76) Inventors: Pragnya J. Desai, San Marcos, CA (US); Paul J. Dunford, San Diego, CA (US); Claudia L. Hofstra, Houten (NL); Lars G. Karlsson, La Jolla, CA (US); Wai-Ping Leung, San Diego, CA (US); Ping Ling, San Diego, CA (US); Robin L. Thurmond, San Diego, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/656,385

(22) Filed: Sep. 5, 2003

Related U.S. Application Data
(60) Provisional application No. 60/408,736, filed on Sep. 6, 2002.
(60) Provisional application No. 60/408,569, filed on Sep. 6, 2002.
(60) Provisional application No. 60/408,579, filed on Sep. 6, 2002.

(51) Int. Cl. C12Q 1/68; G01N 33/574; A61K 31/00

(52) U.S. Cl. 514/1; 435/7.23

(57) ABSTRACT

Methods are disclosed for identifying histamine receptor modulators that affect mast cell or basophil chemotaxis, and the use of such histamine H4 receptor modulators for the prevention, treatment, induction, or other desired modulation of asthma and/or allergic responses, or diseases and/or conditions that are modulated, affected or caused by asthma or allergic responses. Also disclosed is the use of histamine H4 receptor modulators for the prevention, treatment, induction, or other desired modulation of mast cell or basophil chemotactic responses, such as migration to a particular site, or diseases and/or conditions that are modulated, affected or caused by mast cell or basophil chemotaxis.

### CROSS-REFERENCE TO RELATED APPLICATIONS

This claims benefit of U.S. Provisional Application No. 60/408,736, filed Sep. 6, 2002, the entirety of which is incorporated by reference herein. Related applications include U.S. patent application Ser. No. 10/094,357, filed Mar. 8, 2002, U.S. Provisional Application No. 60/408,569, filed Sep. 6, 2002, and U.S. Provisional Application No. 60/408,579, also filed Sep. 6, 2002, the disclosures of which are incorporated in their entireties by reference herein.

### FIELD OF THE INVENTION

The present invention relates to the use of histamine H4 receptor modulators for the prevention, treatment, induction, or other desired modulation of allergic responses, asthma, or diseases and/or conditions that are modulated, affected, or caused by asthma or allergic responses.

### BACKGROUND OF THE INVENTION

Histamine is a multifunctional chemical transmitter that signals through cell surface receptors that are linked to intracellular pathways via guanine nucleotide binding proteins. This class of histamine binding cell surface receptor is part of a broad family of receptors called G-protein coupled receptors or GPCRs. There are currently four subtypes of histamine receptors that have been defined pharmacologically and have been divided into H1, H2, H3, and H4 classifications (Hill et al., Pharmacol. Rev. (1997) 49(3):253-278; Hough, Mol. Pharmacol. (2001) 59:415-419). The H1 histamine receptor has been cloned (Yamashita et al., Proc. Natl. Acad. Sci. U.S.A. (1991) 88(24):11515-11519) and is the target of drugs such as diphenhydramine to block the effects of histamine on smooth muscle in allergic responses. The H2 histamine receptor has been cloned (Gantz et al., Proc. Natl. Acad. Sci. U.S.A. (1991) 88(2):429-433) and is the target of drugs such as ranitidine to block the effects of histamine on acid secretion in the stomach. The H3 histamine receptor, which was hypothesized to exist in 1983 (Arrang et al., Nature (London) (1983) 302(5911):832-837), has been cloned (Lovenberg et al., Mol. Pharmacol. (1999) 55:1101-1107) and is currently a target for development of central nervous system drugs. There are numerous additional functions of histamine in humans which may be mediated by histamine receptors of unknown class, for example, histamine is known to play a role in asthma, yet the current antihistamines that target the H1 and H2 histamine receptor have little, if any, utility in the treatment of asthma (Larsen et al., Pharmacother. (2001) 21:28S-33S).

### SUMMARY OF THE INVENTION

The present invention relates to the use of histamine H4 receptor modulators for the treatment and/or prevention of asthma and/or allergic responses, and the diseases and conditions mediated by asthma and/or allergic responses. Modulators of the histamine H4 receptor may be used for modulating allergic responses in mammals, including the induction, as well as the inhibition, of allergic responses, depending on whether the histamine H4 receptor modulator is an H4 receptor activity agonist, inverse agonist, or antagonist. Asthma and allergic responses mediated by leukocytes, basophils, eosinophils, or mast cells are inhibited by treatment with antagonists or inhibitors of the histamine H4 receptor.

The invention provides in one aspect methods of identifying compounds that modulate mammalian histamine H4 receptor activity, comprising: combining a putative modulator compound of mammalian histamine H4 receptor activity with mammalian histamine H4 receptor and a known histamine receptor H4 ligand; and measuring an effect of the modulator on the H4 receptor protein function, or its ability to bind the ligand, wherein the effect is inhibition, activation, antagonist, agonist or reverse agonist activity, wherein said modulator compound is a modulator of mast cell chemotaxis.

Also provided are monospecific antibodies immunologically reactive with a mammalian histamine H4 receptor protein, wherein said antibody modulates mast cell chemotaxis.

In another aspect, the invention provides a method of identifying compounds that modulate mammalian histamine H4 receptor protein activity, comprising: combining a putative modulator compound of mammalian histamine H4 receptor protein activity with mammalian histamine H4 receptor protein and a known histamine receptor H4 ligand; and measuring an effect of the modulator on the protein function or its ability to bind the ligand, wherein said effect is inhibition, activation, antagonist, agonist or reverse agonist activity, wherein said modulator compound is a modulator of basophil chemotaxis.

Also provided are monospecific antibodies immunologically reactive with a mammalian histamine H4 receptor protein, wherein said antibody modulates basophil chemotaxis in vitro or in vivo.

In another of its several aspects, the invention provides a method of identifying compounds that modulate mammalian histamine H4 receptor-mediated chemotaxis of mast cells to histamine, comprising: in the presence or absence of a histamine H4 receptor modulator, placing mast cells in proximity to histamine under conditions enabling the mast cells to move toward the histamine; and measuring an effect of the histamine H4 receptor modulator on the movement of the mast cells toward the histamine, wherein an increase or decrease in the rate of mast cell movement toward the histamine, or in the number of mast cells that move toward the histamine, is indicative that the test compound modulates histamine H4 receptor-mediated chemotaxis of the mast cells to histamine.

The invention, in another aspect, provides methods of determining if a histamine H4 receptor modulator modulates sub-epithelial accumulation of mast cells in a mammalian trachea in response to exposure to histamine or an allergen, the method comprising: in the presence or absence of pre-treatment with a histamine H4 receptor modulator, exposing a mammal to an aerosol comprising histamine or an allergen under a regimen that would, in the absence of the modulator, result in a pre-determined amount of sub-epithelial mast cell accumulation in the mammal's trachea; and comparing the amount of sub-epithelial mast cell accumulation in the mammal's trachea in the presence and absence of the histamine H4 receptor modulator, a change in the mast cell accumulation in the presence of the modulator as compared to in the absence of the modulator being indicative that the histamine H4 receptor modulator modulates the sub-epithelial accumulation of mast cells in the mammalian trachea in response to the exposure to histamine or an allergen.

Other features and advantages of the present invention will be understood by reference to the figures, detailed description and examples that follow.

### BRIEF DESCRIPTION OF THE DRAWINGS

FIG. 1: Mouse bone marrow-derived mast cell chemotaxis in response to histamine.

FIG. 2: Chemotaxis of mouse bone marrow-derived mast cell in response to histamine can be blocked by a histamine H4 receptor-specific antagonist, but not by H1, H2 or H3 receptor antagonists.

FIG. 3: Chemotaxis of mouse bone marrow-derived mast cells in response to histamine can be blocked by a histamine H4 receptor-specific antagonist with an IC₅₀ of 38 nM.

FIG. 4: In vivo histamine-induced accumulation of mast cells in the mouse trachea can be blocked by a histamine H4 receptor-specific antagonist.

FIG. 5: Differential cell counts in bronchavaeolarlavage (BAL) fluid from the ovalbumin-induced lung inflammation model in mice treated with the H4 receptor antagonist (5-chloro-1H-benzoimidazol-2-yl)-(4-methyl-piperazin-1-yl)-methanone. The p-values from a Student's unpaired t-test are given as follows: * represents p<0.05, ** represents p<0.01 and *** represents p<0.001. FIG. 5A: Symbols used for the treatments are as follows: PBS control, [symbol] OVA (ovalbumin) control, [symbol] OVA+vehicle, [symbol] OVA+H4 antagonist at 50 mg/kg, [symbol] OVA+H4 antagonist at 20 mg/kg, [symbol] OVA+H4 antagonist at 5 mg/kg, [symbol] FIG. 5B: Symbols used for the treatments are as follows: PBS control, [symbol] OVA (ovalbumin) control, [symbol] OVA+vehicle, [symbol] OVA+H4 antagonist at 5 mg/kg, [symbol] OVA+H4 antagonist at 2 mg/kg, [symbol] OVA+H4 antagonist at 0.5 mg/kg,

[symbol]

FIG. 6: Differential cell counts in BAL fluid from the ovalbumin-induced lung inflammation model in mice treated with the H4 receptor antagonist (5-chloro-1H-indol-2-yl)-(4-methyl-piperazin-1-yl)-methanone. The p-values from a Student's unpaired t-test are given as follows: * represents p<0.05, ** represents p<0.01 and *** represents p<0.001. Symbols used for the treatments are as follows: PBS control, [symbol] OVA (ovalbumin) control, [symbol] OVA+vehicle, [symbol] OVA+H4 antagonist at 5 mg/kg, [symbol] OVA+H4 antagonist at 20 mg/kg, [symbol] OVA+H4 antagonist at 50 mg/kg.

FIG. 7: Effects of the H4 receptor antagonist (5-chloro-1H-benzoimidazol-2-yl)-(4-methyl-piperazin-1-yl)-methanone on airway hyper-reactivity in the ovalbumin-induced lung inflammation model in mice (n=8). The p-values from a Student's unpaired t-test are given as follows: * represents p<0.05, ** represents p<0.01 and *** represents p<0.001. Symbols used for the treatments are as follows: PBS control, [symbol] OVA control, [symbol] OVA+H4 antagonist at 20 mg/kg, [symbol] OVA+H4 antagonist at 60 mg/kg, [symbol] OVA+H4 antagonist at 100 mg/kg,

[symbol]

### DETAILED DESCRIPTION OF INVENTION AND PREFERRED EMBODIMENTS

DNA molecules encoding a mammalian histamine H4 receptor have been cloned and characterized and represent members of the class of receptors that couple to G-proteins (Liu et al., (2001) Mol. Pharmacol. (2001) 59:420-426; Liu et al., J. Pharmacol. Exp. Therapeut. (2001) 299(1):121-130). Using a recombinant expression system, functional DNA molecules encoding these histamine H4 receptors have been isolated from mouse, rat, guinea pig, and human. The recombinant protein is useful for a variety of purposes, including, but not limited to, identifying modulators of the human histamine H4 receptor.

The histamine H4 receptors of mouse, rat, and guinea pig have a variety of uses, including, but not limited to, resolving pharmacological differences observed between different mammalian species, particularly since guinea pig, rat, and murine species are commonly used in pre-clinical evaluation of new chemical entities which function as modulators. Such modulators can include for example, agonists, antagonists, and inverse agonists. Modulators identified in the assays disclosed herein are useful, for example, as therapeutic agents, prophylactic agents, and diagnostic agents. Indications for such therapeutic agents include, but are not limited to, asthma, allergy, inflammation, cardiovascular and cerebrovascular disorders, non-insulin-dependent diabetes mellitus, hyperglycemia, constipation, arrhythmia, disorders of the neuroendocrine system, stress, and spasticity, as well as acid secretion, ulcers, airway constriction, and prostate dysfunction. In particular embodiments, modulators that down-regulate the expression, activity or accessibility of H4 receptors are used as therapeutic agents for the treatment of allergic rhinitis and/or asthma. The term “human histamine H4 receptor,” as used herein, refers to protein of the H4 subclass that can function as a specific receptor for histamine.

Allergy and asthma are two of the most common respiratory problems. Allergy is typically characterized by sneezing, runny nose, itchy or watery eyes, and nasal congestion. More severe allergy can be accompanied by additional symptoms of increasing significance. In most cases, allergy is triggered by exposure to an environmental allergen, for example, inhalation of airborne allergens such as pollen, dust mites, mold spores, or animal dander. Other allergens are ingested with food or drink. Like allergy, asthma can be caused by inhalation of allergens but it can also be caused by nonspecific irritants and other factors such as exercise. It differs from allergy in that it is characterized by airway inflammation, bronchial hyper-reactivity and airway obstruction. The symptoms of asthma typically include wheezing, coughing, chest tightness, and shortness of breath.

IgE and mast cells play key roles in both allergy and asthma. Indeed, increases in mast cell number are found in chronic allergic rhinitis and allergy, as well as after exposure to antigens (Crimi et al., Am. Rev. Resp. Dis. (1991) 144:1282; Kirby et al., Am. Rev. Resp. Dis. (1987) 136:379; Slater et al., J. Laryn. Otol. (1996) 110:929; Gauvreau et al., Am. J Resp. Crit. Care Med. (2000) 161:1473; Amin et al. Am. J Resp. Crit. Care Med. (2000) 162:2295; and Kassel et al., Clin. Exp. Allergy (2001) 31:1432). A wide variety of stimuli may cause the activation of mast cells, and subsequently cause them to migrate to a particular location (recruitment) and/or to undergo degranulation. These stimuli may be immunologic (such as antigens or allergens) or non-immunologic (such as chemical agents) in nature. Activated mast cells release a number of inflammatory mediators, such as histamine, which are involved in the acute-phase responses, including increased vascular permeability, bronchoconstriction, vasodilation, and recruitment of inflammatory cells. The influx of inflammatory cells in turn leads to the release of additional mediators that facilitate and prolong the response. It is this chronic inflammatory process that leads to the tissue remodeling associated with both conditions. Recently, the histamine receptor H4 has been cloned and demonstrated to be expressed in a variety of cells, including, but not limited to, mast cells.

Another important cell type is basophils, which, in contrast to mast cells that normally reside in tissues that interface directly with the environment, are found circulating in the blood. In response to inflammatory stimuli, basophils migrate to the site of inflammation. Like mast cells, they respond to IgE or other agents and release inflammatory mediators, such as histamine. In both allergic rhinitis and allergy there is an increase in the number of basophils in the airways where they are thought to play a role in the late phase responses (Kirby et al. (1987), supra; Gauvreau et al. (2000), supra; and Braunstahl et al., Am. J Resp. Crit. Care Med. (2001) 164:858).

In general, allergy can be controlled for example by antihistamines (all H1 receptor antagonists) and decongestants, however, there is still a need for disease-modifying agents. The same is true for asthma, where inhaled bronchodilators and steroids are the major therapies. Of note is the fact that, although histamine and mast cells are thought to be important for both conditions, the currently available H1 and H2 receptor antagonists are only useful in the treatment of allergy and have little, if any, benefit in asthma This suggests that, for asthma, other histamine receptors, such as the H4 receptor, play a role.

Numerous medical texts are published and are available to those of skill in the relevant art fields. In addition, numerous scientific and medical research publications have been published in the fields of allergy and asthma. Examples of available published textbooks on the subject of inflammation include Barnes et al., Asthma and COPD: Basic Mechanisms and Clinical Management, (Academic Press, London, 2002); Eric et al. Bronchial Asthma: Principles of Diagnosis and Treatment, (Humana Press, 2001); Mygind, Allergic and Non-Allergic Rhinitis: Clinical Aspects, (W.B. Saunders Company, 1993); Grammer and Greenberger, Patterson's Allergic Diseases, (Lippincott Williams & Wilkins Publishers, 2002); Cecil et al., Textbook Of Medicine, 18th Ed. (W.B. Saunders Company, 1988); and Steadmans Medical Dictionary.

The present invention demonstrates that the histamine H4 receptor is involved in asthma and allergic responses, and particularly involved in mast cell or basophil recruitment to the site of stimulus, and that antagonists for this receptor are effective anti-asthma and/or anti-allergy agents. The present invention provides in certain presently preferred embodiments methods for modulating asthma or allergic responses that are directly or indirectly mediated by the histamine H4 receptor. In one of its aspects, the present invention also provides methods for inhibiting, preventing, ameliorating, inducing, or otherwise affecting asthma or allergic responses that are mediated by the histamine H4 receptor, through the treatment of a mammal with modulators of the histamine H4 receptor. Modulators of the histamine H4 receptor that are useful in the method of the present invention include, but are not limited to, antibodies and antibody fragments that bind the histamine H4 receptor, RNAi, antisense, or other agents that modulate expression of genes encoding the histamine H4 receptor, and inhibitors, activators, antagonists, agonists and reverse agonists of the histamine H4 receptor, including, but not limited to, proteins, nucleic acids, or other organic molecules. These modulators are useful for administration to humans in need thereof, and are also useful for veterinary purposes to administer to non-human animals, including, but not limited to, non-human mammals.

Monospecific antibodies to mammalian histamine H4 receptor are purified from mammalian antisera containing antibodies reactive against mammalian histamine H4 receptor or are prepared as monoclonal antibodies reactive with mammalian histamine H4 receptor using the technique of G. Kohler and C. Milstein (Nature (1975) 256:495-497). Monospecific antibody, as used herein, is defined as a single antibody species or multiple antibody species with homogenous binding characteristics for mammalian histamine H4 receptor. Homogenous binding, as used herein, refers to the ability of the antibody species to bind to a specific antigen or epitope, such as those associated with the mammalian histamine H4 receptor, as described above. Methods for preparing monospecific polyclonal and monoclonal antibodies are routine in the art.

It is also readily apparent to those skilled in the art that the well-known methods for producing monospecific antibodies may be utilized to produce antibodies specific for mammalian histamine H4 receptor polypeptide fragments, or full-length nascent mammalian histamine H4 receptor polypeptide, or the individual mammalian histamine H4 receptor epitopes. Specifically, it is readily apparent to those skilled in the art that monospecific antibodies may be generated that are specific for a portion of only one species of mammalian histamine H4 receptor or the fully functional histamine H4 receptor. It is also readily apparent to one of ordinary skill in the art that antibodies that are specific for the histamine H4 receptor may cause a change in the functional activity of the receptor, including, but not limited to, causing the receptor to be activated or inactivated, blocked from binding its ligand, blocked from releasing its bound ligand, or prevented from functioning in the normal fashion associated with a histamine H4 receptor.

Nucleotide sequences that are complementary to the human or other mammalian histamine H4 receptor encoding DNA sequence can be synthesized for antisense therapy. These antisense molecules may be DNA, stable derivatives of DNA such as phosphorothioates or methylphosphonates, RNA, stable derivatives of RNA such as 2′-O-alkylRNA, or other human histamine H4 receptor antisense oligonucleotide mimetics. Human histamine H4 receptor antisense molecules may be introduced into cells by microinjection, liposome encapsulation or by expression from vectors harboring the antisense sequence. Human histamine H4 receptor antisense therapy may be particularly useful for the treatment of diseases where it is beneficial to reduce human histamine H4 receptor activity.

Human or other mammalian histamine H4 receptor gene therapy may be used to introduce the histamine H4 receptor into the cells of target organisms. The histamine H4 receptor gene can be ligated into viral vectors that mediate transfer of the human histamine H4 receptor DNA by infection of recipient host cells. Suitable viral vectors include retrovirus, adenovirus, adeno-associated virus, herpes virus, vaccinia virus, poliovirus and the like. Alternatively, human histamine H4 receptor DNA can be transferred into cells for gene therapy by non-viral techniques, including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo human histamine H4 receptor gene therapy. Human histamine H4 receptor gene therapy may be particularly useful for the treatment of diseases where it is beneficial to elevate human histamine H4 receptor activity.

Histamine is a biogenic amine transmitter that functions in some capacity in nearly all physiological and pathophysiological situations. Histamine acts as a neurotransmitter and neuromodulator in the central nervous system, mediates asthma and allergic responses, regulates airway function, controls acid secretion in the stomach, regulates cardiovascular function, as well as arterial and venous responses, and is likely to be involved in processes yet to be determined. The histamine receptors that mediate these effects are not completely characterized. One way to understand which histamine receptors are involved in these processes is to develop chemical modulators (such as agonists, antagonists, and inverse agonists) of the receptors as research tools and therapeutic entities. Recombinant host cells expressing the mammalian histamine H4 receptor can be used to provide materials for a screening method to identify such agonists and antagonists. As such, this invention provides a way to identify new agonists and antagonists of the histamine H4 receptor that may prove useful as research tools or may be used as therapeutics to treat disorders directly or indirectly involving histamine receptors, such as allergic responses and asthma Assays to detect compound interaction or modulation of the histamine H4 receptor include, but are not limited to, direct ligand binding assays, competitive (or displacement) ligand binding assays, or functional assays that measure the response of the receptor to the ligand, for example, by production of cAMP or, as in a preferred embodiment, mast cell or basophil chemotaxis. Although assays of this general type are well known to those skilled in the art, they were previously not possible prior to obtaining the recombinant molecules taught herein, nor were they appreciated as having practical utility prior to the inventors' discovery of the physiological effect of blocking the H4 receptor, e.g., inhibition of mast cell chemotaxis, among other effects.

An exemplary competitive binding assay involves the following steps:

1. Mammalian cultured cells are transiently transfected with a nucleic acid molecule encoding a histamine H4 receptor, and thereafter grown in culture.

2. Cell membranes are prepared from the transfected cells by homogenization of cells and separation of the membrane fraction, e.g., by centrifugation.

3. For controls, cell membranes are incubated with detectably labeled histamine (for example, tritiated histamine) in the presence or absence of excess histamine.

4. For test samples, cell membranes are incubated with detectably labeled histamine as above, in the presence of various concentrations of the compound(s) to be tested.

5. Kᵢ values are calculated according to known methods.

Certain embodiments of the invention provide in vitro assays for measuring the effect of a histamine H4 modulator on chemotaxis of mast cells or basophils to histamine. For these assays, as applied to mast cells, bone marrow is obtained from an animal source (e.g., mice), and cultured for an appropriate time in a medium that promotes differentiation of mast cells. Segmented culture wells of appropriate pore size are coated with fibronectin. After removal of the fibronectin, culture medium containing histamine is added to the bottom chambers of the segmented wells. To the top wells are added various concentrations of the compounds to be tested, along with an aliquot of mast cells. The wells are incubated under conditions enabling migration of the mast cells from the top chambers to the bottom chambers. Following incubation, numbers of cells in the bottom chambers are counted, e.g., by flow cytometry.

Additionally, in another of its several aspects the invention provides in vivo animal models for assessing the effect of a histamine H4 modulator on histamine- or allergen-induced sub-epidermal accumulation of mast cells in the trachea. Test animals (e.g., mice) are exposed to an aerosol comprising saline (control) or histamine for a short period (e.g., 20 minutes) on a few (e.g., two) consecutive days. For test animals, histamine aerosolized mice are pre-dosed prior to each aerosol with either saline or a compound to be tested. After the treatments, animals are sacrificed and a section of the trachea is removed and sectioned. Mast cells are quantitatively detected by selective staining, e.g., immunohistochemically or with toluidine blue. Mast cells may be further quantitated as sub-mucosal or sub-epithelial depending on their location within the tracheal section. Migration of mast cells into the sub-epithelial space is indicative of an allergic response.

Preferred methods of the present invention are used to identify chemical compounds that act, for example, as agonists, antagonists or inverse agonists of the histamine H4 receptor. As described in greater detail in Example 1, a screening assay involving binding of the H4 receptor to candidate compounds has identified several classes of compounds that bind to the receptor. Binding affinity of these compounds has been positively correlated with the ability of the compounds to inhibit chemotaxis of mast cells to histamine. An exemplary compound has been further tested in an in vivo model, where it has been demonstrated that the compound reduces or inhibits histamine-mediated migration of mast cells into the sub-epithelial space of the trachea. Such movement is similar to what is thought to occur upon allergen exposure.

Accordingly, in addition to methods of identifying modulators of the histamine H4 receptor, presently preferred embodiments of the present invention provide modulators of the H4 receptor identified by the various screening assays described herein. These modulators bind to the recombinant H4 receptor in vitro. In a preferred embodiment, they possess a Kᵢ for the receptor, under conditions defined herein, of less than 1 μM, more preferably less than 900 nM, 800 nM, 700 nM, or 600 nM, respectively, in ascending order of preference. In more preferred embodiments, they possess a Kᵢ for the receptor of less than 500 nM, even more preferably less than 400 nM, yet more preferably less than 300 nM, and even more preferably less than 200 nM. In particularly preferred embodiments, the Kᵢ is less than 100 nM, 50 nM, 40 nM, 30 nM, 20 nM and 10 nM, respectively, in ascending order of preference. Inasmuch as the Kᵢ of such compounds has been demonstrated to be positively correlated with the ability of the compound to inhibit histamine-mediated mast cell chemotaxis in vitro, it will be appreciated by one of skill in the art that compounds with greater binding affinity may be used to particular advantage as therapeutic agents in the treatment of pathological conditions associated with H4-mediated signal transduction, including, but not limited to, allergy and asthma. In accordance with the present invention, several classes of compounds have been demonstrated to possess the requisite binding and chemotaxis-modulating features mentioned above, as described in greater detail in the examples that follow.

In one of its several aspects, the present invention is also directed to methods for screening for compounds that modulate the expression of DNA or RNA encoding mammalian histamine H4 receptor as well as the function of mammalian histamine H4 receptor protein in vitro and in vivo. Compounds that modulate these activities may be DNA, RNA, peptides, proteins, or non-proteinaceous organic molecules. Compounds may modulate by increasing or attenuating the expression of DNA or RNA encoding mammalian histamine H4 receptor, or the function of mammalian histamine H4 receptor protein. Compounds that modulate the expression of DNA or RNA encoding mammalian histamine H4 receptor or the function of mammalian histamine H4 receptor protein may be detected by a variety of assays. The assays may be a simple “yes/no” assay to determine whether there is a change in expression of nucleic acid encoding the receptor, or a change in the function or activity of the receptor protein. The assay may be made quantitative by comparing the expression or function of a test sample with the levels of receptor expression or receptor protein function in a standard sample. Modulators identified in this process are useful as therapeutic agents, research tools, and diagnostic agents.

Pharmaceutically useful compositions comprising modulators of mammalian histamine H4 receptor activity, via receptor binding or other mechanisms as taught herein, may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the modulator or other biologically active agent.

Therapeutic or diagnostic compositions of the invention are administered to an individual in amounts sufficient to treat or diagnose disorders in which modulation of mammalian histamine H4 receptor-related activity is indicated. The effective amount may vary according to a variety of factors such as the individual's condition, weight, sex and age. Other factors include the mode of administration. The pharmaceutical compositions may be provided to the individual by a variety of routes such as subcutaneous, topical, oral, intranasal and intramuscular.

The term “chemical derivative” describes a molecule that contains additional chemical substituents or moieties that are not normally a part of the base molecule. Such moieties may improve the solubility, half-life, absorption, etc. of the base molecule. Alternatively, the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages defined by routine testing in order to obtain optimal inhibition of the mammalian histamine H4 receptor or its activity while minimizing any potential toxicity. In addition, co-administration or sequential administration of other agents may be desirable.

The present invention, in another of its several aspects, also provides suitable topical, oral, systemic and parenteral pharmaceutical formulations for use in the methods of treatment of the present invention. The compositions containing compounds or modulators identified according to this invention as the active ingredient for use in the modulation of mammalian histamine H4 receptor receptors can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a mammalian histamine H4 receptor-modulating agent.

The daily dosage of the products may be varied over a wide range from 0.01 to 1,000 mg per patient, per day. For oral administration, the compositions are preferably provided in the form of scored or un-scored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the mammalian histamine H4 receptor modulators are adjusted when combined to achieve desired effects. On the other hand, dosages of these various agents may be independently optimized and combined to achieve a synergistic result wherein the pathology is reduced more than it would be if either agent were used alone.

For delivery to the airway of a patient in need of treatment, the compositions are preferably formulated as an aerosol, which may be delivered nasally or by means of an inhaler, as is well known in the art. The amounts of active ingredient are adjusted for this form of delivery according to standard methods.

Advantageously, compounds or modulators of the present invention may be administered in a single daily dose, or the total daily dosage may be administered in divided doses of two, three or four times daily. Furthermore, in addition to oral and intranasal/inhalatory delivery, compounds or modulators for the present invention can be administered via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds or modulators of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

In preferred methods of the present invention, an active drug component or ingredient can comprise one or more compounds or modulators herein described, and can be preferably administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of tablets or capsules, active drug component(s) can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars, such as glucose or beta-lactose, corn sweeteners, natural and synthetic gums, such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methylcellulose, agar, bentonite, xanthan gum and the like.

For liquid forms, active drug components can be combined in suitably flavored suspending or dispersing agents, such as the synthetic and natural gums, for example, tragacanth, acacia, methyl-cellulose and the like. Other dispersing agents that may be employed include glycerin and the like. For parenteral administration, sterile suspensions and solutions are preferred. Isotonic preparations that generally contain suitable preservatives are preferred when intravenous administration is desired.

Topical preparations containing an active drug component can be admixed with a variety of carrier materials well known in the art, such as, e.g., alcohols, aloe vera gel, allantoin, glycerin, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

The compounds or modulators of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds or modulators of the present invention may also be coupled with soluble polymers as targetable drug carriers. Such polymers can include polyvinylpyrrolidone, pyran copolymer, polyhydroxypropylmethacryl-amidephenol, polyhydroxy-ethylaspartamidephenol, or polyethyl-eneoxidepolylysine substituted with palmitoyl residues. Furthermore, the compounds or modulators of the present invention may be coupled to a class of biodegradable polymers useful in achieving controlled release of a drug, for example, polylacetic acid, polyepsilon caprolactone, polyhydroxy butyric acid, polyorthoesters, polyacetals, polydihydropyrans, polycyanoacrylates and cross-linked or amphipathic block copolymers of hydrogels.

For oral administration, the compounds or modulators may be administered in capsule, tablet, or bolus form or alternatively they can be mixed in the animal's feed. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the compounds or modulators and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated and the type and severity of the symptoms. The active ingredient may also be administered as an additive to the feed by simply mixing the compound with the feedstuff or by applying the compound to the surface of the feed. Alternatively the active ingredient may be mixed with an inert carrier and the resulting composition may then either be mixed with the feed or fed directly to the animal. Suitable inert carriers include corn meal, citrus meal, fermentation residues, soya grits, dried grains and the like. The active ingredients are intimately mixed with these inert carriers by grinding, stirring, milling, or tumbling such that the final composition contains from 0.001 to 5% by weight of the active ingredient.

The compounds or modulators may alternatively be administered parenterally via injection of a formulation consisting of the active ingredient dissolved in an inert liquid carrier. Injection may be either intramuscular, intra-ruminal, intratracheal, or subcutaneous. The injectable formulation consists of the active ingredient mixed with an appropriate inert liquid carrier. Acceptable liquid carriers include the vegetable oils, such as peanut oil, cottonseed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the active ingredient in the liquid carrier such that the final formulation contains from 0.005 to 10% by weight of the active ingredient.

Topical application of the compounds or modulators is possible through the use of a liquid drench or a shampoo containing the instant compounds or modulators as an aqueous solution or suspension. These formulations generally contain a suspending agent such as bentonite and normally will also contain an antifoaming agent. Formulations containing from 0.005 to 10% by weight of the active ingredient are acceptable. Preferred formulations are those containing from 0.01 to 5% by weight of the instant compounds or modulators.

The following examples are provided for the purpose of illustrating the present invention without, however, limiting the same thereto.

### EXAMPLE 1

### Cloning of Human Histamine H4 Receptor cDNA into a Mammalian Expression Vector

The human histamine H4 receptor cDNAs (collectively referred to as pH4R) were cloned into the mammalian expression vector pCIneo. The human histamine H4 receptor cDNA clone was isolated from the human thalamus cDNA library. The full-length cDNA was used as the template for PCR using specific primers with EcoR1 and Not1 sites for cloning. The PCR product was purified on a column (Wizard PCR DNA purification kit from Promega) and digested with Not1 and EcoR1 (NEB) to create cohesive ends. The product was purified by a low melting agarose gel electrophoresis. The pCIneo vector was digested with EcoR1 and Not1 enzymes and subsequently purified on a low melt agarose gel. The linear vector was used to ligate to the human histamine H4 receptor cDNA inserts. Recombinants were isolated, designated human histamine H4 receptor, and used to transfect mammalian cells (SK—N-MC cells) by CaPO₄-DNA precipitation. Stable cell clones were selected by growth in the presence of G418. Single G418 resistant clones were isolated and shown to contain the intact human histamine H4 receptor gene. Clones containing the human histamine H4 receptor cDNAs were analyzed for pH4R expression by measuring inhibition of adenylate cyclase in response to histamine according to the method of Konig et al. (Mol. Cell. Neurosci. (1991) 2(4):331-337) or by directly measuring cAMP accumulation by radioimmunoassay using Flashplates (NEN). Expression was also analyzed using [³H]-histamine binding assays (Clark et al., Eur. J Pharmacol. (1992) 210(1):31-35). Recombinant plasmids containing human histamine H4 receptor encoding DNA were used to transform the mammalian COS7 or CHO cells or HEK293 or L-cells or SK—N-MC cells.

Cells expressing human histamine H4 receptor, stably or transiently, were used to test for expression of human histamine H4 receptor and for [³H]-histamine binding activity. These cells were used to identify and examine other compounds for their ability to modulate, inhibit or activate the human histamine H4 receptor and to compete for radioactive histamine binding.

Cassettes containing the human histamine H4 receptor cDNA in the positive orientation with respect to the promoter were ligated into appropriate restriction sites 3′ of the promoter and identified by restriction site mapping and/or sequencing. These cDNA expression vectors were introduced into fibroblastic host cells, for example COS-7 (ATCC# CRL1651), and CV-1 tat [Sieckevitz et al., Science (1987) 238:1575-1578], 293, L (ATCC# CRL6362), SK—N-MC (ATCC# HTB-10), by standard methods including, but not limited to, electroporation or chemical procedures (cationic liposomes, DEAE dextran, calcium phosphate). Transfected cells and cell culture supernatants were harvested and analyzed for human histamine H4 receptor expression as described herein.

All of the vectors used for mammalian transient expression can be used to establish stable cell lines expressing human histamine H4 receptor. Unaltered human histamine H4 receptor cDNA constructs cloned into expression vectors are expected to program host cells to make human histamine H4 receptor protein. The transfection host cells include, but are not limited to, CV-1-P [Sieckevitz et al., supra], tk-L [Wigler et al., Cell (1977) 11(1):223-232], NS/0, and dHFr-CHO [Randall J. Kaufman and Phillip A. Sharp, J. Mol. Biol. (1982) 159:601-621].

Co-transfection of any vector containing human histamine H4 receptor cDNA with a drug selection plasmid including, but not limited to, G418, aminoglycoside phosphotransferase; hygromycin, hygromycin-B phosphotransferase; APRT, xanthine-guanine phosphoribosyl-transferase, will allow for the selection of stably transfected clones. Levels of human histamine H4 receptor are quantitated by the assays described herein.

Human histamine H4 receptor cDNA constructs were also ligated into vectors containing amplifiable drug-resistance markers for the production of mammalian cell clones synthesizing the highest possible levels of human histamine H4 receptor. Following introduction of these constructs into cells, clones containing the plasmid were selected with the appropriate agent, and isolation of an over-expressing clone with a high copy number of plasmids was accomplished by selection in increasing doses of the agent.

The expression of recombinant human histamine H4 receptor was achieved by transfection of full-length human histamine H4 receptor cDNA into a mammalian host cell.

Characterization of Human Histamine H4 Receptor

Human SK—N-MC cells were transfected with pH4R and selected in the presence of neomycin for ten days. Individual colonies were picked and grown in six-well dishes. Cells were then plated onto 96-well plates and grown to confluence. Cells were incubated for 20 min with isobutylmethylxanthine (1 mM). Cells were stimulated with histamine (100 pM-100 μM) for 5 min. Cells were then stimulated with forskolin (3 μM) and allowed to incubate at 37° C. for 20 min. Cells were treated with 0.1 N HCl. Cells were frozen and thawed. Aliquots of the supernatant were analyzed for their cyclic AMP content using a standard cAMP radioimmunoassay kit (Flashplates, NEN). The forskolin treatment raises the intracellular concentration of cAMP. Any cells that responded to histamine by decreasing the cAMP content in response to forskolin were considered to be expressing active functional human histamine H4 receptor. The recombinant human histamine H4 receptor expressed from the human histamine H4 receptor-encoding DNA molecule described herein was shown to be specifically activated by histamine.

### EXAMPLE 2

### Binding Assay on Recombinant Human Histamine H4 Receptor

SK—N-MC cells or COS7 cells were transiently transfected with pH4R and grown in 150 cm² tissue culture dishes. Cells were washed with saline solution, scraped with a cell scraper and collected by centrifugation (1000 rpm, 5 min). Cell membranes were prepared by homogenization of the cell pellet in 20 mM Tris-HCl with a polytron tissue homogenizer for 10 sec at high speed. The homogenate was centrifuged at 1000 rpm for 5 min at 4° C. The supernatant was then collected and centrifuged at 20,000×g for 25 min at 4° C. The final pellet was resuspended in 50 mM Tris-HCl. Cell membranes were incubated with ³H-histamine (5-70 nM) in the presence or absence of excess histamine (10000 nM). Incubation occurred at room temperature for 45 min. Membranes were harvested by rapid filtration over Whatman GF/C filters and were washed 4 times with ice-cold 50 mM Tris HCl. The filters were then dried, mixed with scintillant and counted for radioactivity. SK—N-MC or COS7 cells expressing human histamine H4 receptor were used to measure the affinity of binding of other compounds and their ability to displace ³H-ligand binding by incubating the above-described mixture in the presence of various concentrations of inhibitor or compound to be tested. For competition binding studies using ³H-histamine, Kᵢ values were calculated, based on an experimentally determined K_{D} value of 5 nM and a ligand concentration of 5 nM, according to Cheng and Prusoff (Biochem. Pharmacol. (1973) 22:3099-3108): Kᵢ=(IC₅₀)/(1+([L]/(K_{D})).

Results of competition binding studies are set forth in Table 1 for various compounds within three classes of compounds, which may be prepared as described in U.S. patent application Ser. No. 10/094,357 (see also International Publication No. WO 02/072548), U.S. Provisional Application No. 60/408,569, and Provisional Application No. 60/408,723, the disclosures of which are incorporated by reference herein.

One class comprises the headgroup

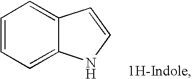

and is referred to herein as the “indole” class.

Another class comprises one of the following headgroups, and is referred to herein as the “bicyclic pyrrole” class:

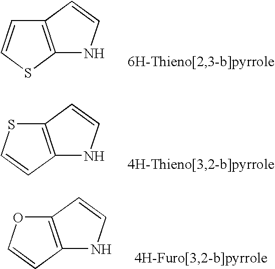

The third class comprises the headgroup

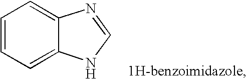

and is referred to herein as the “benzoimidazole” class

TABLE 1

| COMPOUND | Kᵢ (nM) |
|---|---|
| (5-Chloro-7-methyl-1H-indol-2-yl)-(4-methyl-piperazin-1-yl)-methanone | 1 |
| (2-Chloro-3-methyl-4H-thieno[3,2-b]pyrrol-5-yl)-(4-methyl-piperazin-1-yl)-methanone | 3 |
| (5-Chloro-1H-indol-2-yl)-(4-methyl-piperazin-1-yl)-methanone | 5 |
| (2,3-Dimethyl-4H-thieno[3,2-b]pyrrol-5-yl)-(4-methyl-piperazin-1-yl)-methanone | 5 |
| (2,3-Dichloro-4H-thieno[3,2-b]pyrrol-5-yl)-(4-methyl-piperazin-1-yl)-methanone | 5.5 |
| (7-Methyl-1H-indol-2-yl)-(4-methyl-piperazin-1-yl)-methanone | 6.6 |
| (7-Amino-1H-indol-2-yl)-(4-methyl-piperazin-1-yl)-methanone | 7 |
| (5-Bromo-1H-indol-2-yl)-(4-methyl-piperazin-1-yl)-methanone | 8 |
| (5,7-Dichloro-1H-indol-2-yl)-(4-methyl-piperazin-1-yl)-methanone | 10 |
| (5-Chloro-1H-indol-2-yl)-piperazin-1-yl-methanone | 10 |
| (2,3-Dichloro-6H-thieno[2,3-b]pyrrol-5-yl)-(4-methyl-piperazin-1-yl)-methanone | 10 |
| (5-Methyl-1H-indol-2-yl)-(4-methyl-piperazin-1-yl)-methanone | 11 |
| (4,5-Dichloro-1H-indol-2-yl)-(4-methyl-piperazin-1-yl)-methanone | 11 |
| (4-Methyl-piperazin-1-yl)-(5-trifluoromethyl-1H-benzoimidazol-2-yl)-methanone | 11 |
| (5-Fluoro-1H-indol-2-yl)-(4-methyl-piperazin-1-yl)-methanone | 18 |
| (5,7-Difluoro-1H-indol-2-yl)-(4-methyl-piperazin-1-yl)-methanone | 19 |
| (5-Amino-1H-indol-2-yl)-(4-methyl-piperazin-1-yl)-methanone | 19 |
| (5-Hydroxy-1H-indol-2-yl)-(4-methyl-piperazin-1-yl)-methanone | 19 |
| (4-Methyl-piperazin-1-yl)-(3-methyl-4H-thieno[3,2-b]pyrrol-5-yl)-methanone | 21 |
| (7-Chloro-1H-indol-2-yl)-(4-methyl-piperazin-1-yl)-methanone | 23 |
| (5-Chloro-1H-indol-2-yl)-[4-(2-hydroxy-ethyl)-piperazin-1-yl]-methanone | 25 |
| (2-Chloro-6H-thieno[2,3-b]pyrrol-5-yl)-(4-methyl-piperazin-1-yl)-methanone | 25 |
| (5-Chloro-1H-benzoimidazol-2-yl)-(4-methyl-piperazin-1-yl)-methanone | 25 |
| (5-Fluoro-1H-benzoimidazol-2-yl)-(4-methyl-piperazin-1-yl)-methanone | 26 |
| (5-Chloro-1H-indol-2-yl)-(3,4-dimethyl-piperazin-1-yl)-methanone | 27 |
| (5,6-Difluoro-1H-benzoimidazol-2-yl)-(4-methyl-piperazin-1-yl)-methanone | 28 |
| (5,7-Dimethyl-1H-indol-2-yl)-(4-methyl-piperazin-1-yl)-methanone | 30 |
| (2-Chloro-3-methyl-4H-thieno[3,2-b]pyrrol-5-yl)-piperazin-1-yl-methanone | 30 |
| (4-Methyl-1H-benzoimidazol-2-yl)-(4-methyl-piperazin-1-yl)-methanone | 31 |
| (1H-Benzoimidazol-2-yl)-(4-methyl-piperazin-1-yl)-methanone | 32 |
| (6-Hydroxy-1H-indol-2-yl)-(4-methyl-piperazin-1-yl)-methanone | 32.5 |
| (5-Chloro-1H-indol-2-yl)-((R)-3-methyl-piperazin-1-yl)-methanone | 34 |
| (5-Chloro-1H-indol-2-yl)-((S)-3-methyl-piperazin-1-yl)-methanone | 36 |
| (4-Bromo-1H-indol-2-yl)-(4-methyl-piperazin-1-yl)-methanone | 40 |
| (2-Chloro-4H-thieno[3,2-b]pyrrol-5-yl)-(4-methyl-piperazin-1-yl)-methanone | 40 |
| (5-Chloro-1H-indol-2-yl)-(3-methyl-piperazin-1-yl)-methanone | 41 |
| (5-Fluoro-1H-benzoimidazol-2-yl)-piperazin-1-yl-methanone | 42 |
| (7-Amino-1H-indol-2-yl)-piperazin-1-yl-methanone | 43 |
| (4-Methyl-piperazin-1-yl)-(5-nitro-1H-indol-2-yl)-methanone | 46 |
| (7-Hydroxy-1H-indol-2-yl)-(4-methyl-piperazin-1-yl)-methanone | 47 |
| (6-Chloro-5-fluoro-1H-benzoimidazol-2-yl)-(4-methyl-piperazin-1-yl)-methanone | 53 |
| (7-Bromo-1H-indol-2-yl)-(4-methyl-piperazin-1-yl)-methanone | 55 |
| (2-Chloro-6H-thieno[2,3-b]pyrrol-5-yl)-piperazin-1-yl-methanone | 56 |
| (3-Bromo-4H-thieno[3,2-b]pyrrol-5-yl)-(4-methyl-piperazin-1-yl)-methanone | 56 |
| (3-Methyl-4H-thieno[3,2-b]pyrrol-5-yl)-piperazin-1-yl-methanone | 80 |
| (4-Methyl-piperazin-1-yl)-(6H-thieno[2,3-b]pyrrol-5-yl)-methanone | 85 |
| (5-Chloro-1H-benzoimidazol-2-yl)-piperazin-1-yl-methanone | 87 |
| 1H-Benzoimidazole-2-carboxylic acid(8-methyl-8-aza-bicyclo[3.2.1]oct-3-yl)-amide | 89 |
| (5-Bromo-benzofuran-2-yl)-(4-methyl-piperazin-1-yl)-methanone | 95 |
| (1H-Indol-2-yl)-(3-methyl-piperazin-1-yl)-methanone | 100 |
| (1H-Indol-2-yl)-(4-methyl-piperazin-1-yl)-methanone | 117 |
| (6-Chloro-1H-indol-2-yl)-(4-methyl-piperazin-1-yl)-methanone | 124 |
| (4-Methyl-piperazin-1-yl)-(4H-thieno[3,2-b]pyrrol-5-yl)-methanone | 125 |
| (1H-Indol-2-yl)-(4-methyl-piperazin-1-yl)-methanethione | 132 |
| (4-Methyl-1H-benzoimidazol-2-yl)-piperazin-1-yl-methanone | 135 |
| (2,3-Dimethyl-4H-furo[3,2-b]pyrrol-5-yl)-(4-methyl-piperazin-1-yl)-methanone | 140 |
| [5-(3-Methoxy-phenyl)-1H-indol-2-yl]-(4-methyl-piperazin-1-yl)-methanone | 145 |
| (4-Methyl-1H-benzoimidazol-2-yl)-(3-methyl-piperazin-1-yl)-methanone | 156 |
| (3-Methyl-piperazin-1-yl)-(3-methyl-4H-thieno[3,2-b]pyrrol-5-yl)-methanone | 161 |
| (2-Chloro-6H-thieno[2,3-b]pyrrol-5-yl)-(hexahydro-pyrrolo[1,2-a]pyrazin-2-yl)-methanone | 176 |
| (6-Bromo-1H-indol-2-yl)-(4-methyl-piperazin-1-yl)-methanone | 188 |
| 5-Methyl-1H-benzoimidazole-2-carboxylic acid(8-methyl-8-aza-bicyclo[3.2.1]oct-3-yl)- | 613 |
| amide | |
| (3-Bromo-4H-thieno[3,2-b]pyrrol-5-yl)-(3-methyl-piperazin-1-yl)-methanone | 980 |

### EXAMPLE 3

### Ligand Binding to Mammalian Histamine H4 Receptors

The affinity of ³H-histamine for rat, mouse, guinea pig, and human histamine H4 receptors was determined using standard techniques as described herein. Saturation binding was performed on membranes from SK—N-MC cells stably transfected with the appropriate histamine H4 receptor. The KD values were derived from a −1/slope of the linear regression of a Scatchard plot (bound/free vs. bound). The results are shown in Table 2.

TABLE 2

| Species | ³H-histamine K_{d} (nM) |
|---|---|
| Rat | 105 |
| Murine | 34 |
| Guinea Pig | 20 |
| Human | 5 |

The relative affinity of several known histamine receptor ligands was determined by competitive binding of 30 nM ³H-histamine. Kᵢ values for each ligand were calculated according to the method of Cheng and Pruscoff (Kᵢ=IC₅₀/(1+[³H-histamine]/K_{d}). The K_{D} values for ³H-histamine were those set forth in Table 2. The results are presented in Table 3.

TABLE 3

| | Human Kᵢ | Guinea Pig Kᵢ | Rat Kᵢ | Murine |
|---|---|---|---|---|
| Compound | (nM) | (nM) | (nM) | Kᵢ (nM) |
| Imetit | 1.3 | 30 | 6.8 | 6.6 |
| Histamine | 5.9 | 27 | 70 | 41 |
| Clobenpropit | 4.9 | 3.6 | 63 | 14 |
| N-methylhistamine | 48 | 220 | 552 | 303 |
| Thioperamide | 52 | 83 | 28 | 22 |
| R-α-methylhistamine | 144 | 486 | 698 | 382 |
| Burimamide | 124 | 840 | 958 | 696 |
| Clozapine | 626 | 185 | 2200 | 2780 |

### EXAMPLE 4

### Inhibition of Histamine-Induced Mast Cell Chemotaxis in vitro by Histamine H4 Receptor Antagonists

This example demonstrates the discovery for the first time that histamine H4 receptor antagonists can block the chemotactic response of mast cells in response to a stimulus.

Methods

Bone Marrow Mast Cell Culture

Mast cells were differentiated from bone marrow derived from BALB/c or C57bl/6j mice. Mice were sacrificed by asphyxiation under 95% CO₂ and femurs were removed. Bone marrow was aseptically isolated from the femurs. The cells (5×10⁵/mL) were cultured at 37° C. with 5% CO₂ in culture medium consisting of RPMI with 10% FCS, 0.1 mM non-essential amino acids, 50 μg/mL penicillin/streptomycin and 20% conditioned WEHI-3 medium. Conditioned WEHI-3 medium was prepared from WEHI-3 cells (ATCC), which were cultured in Iscoves Dulbeccos medium with 10% FCS, 4 mM L-glutamine, 1.5 g/L sodium carbonate, 0.05 μM beta-mercaptoethanol and 50 μg/mL penicillin/streptomycin. The filtered supernatant was used as the conditioned WEHI-3 medium. After 16 h in culture, the bone marrow cells were transferred to a new flask. The medium was refreshed once per week. After four weeks, the cells were tested by flow cytometry for IgE receptor and CD117 (c-kit) expression. Mast cells were incubated with anti-DNP IgE (ICN Pharmaceuticals, Costa Mesa, Calif.) or vehicle for 30 min, followed by FITC labeled anti-IgE (Pharmingen) or FITC labeled CD117 (Pharmingen) for 30 min on ice. The cultured mast cells consisted of a homogeneous population which was >99% IgE receptor positive and >99% CD117 positive. Mast cells of four to eight weeks culture time were used for experiments.

Chemotaxis Assay

Transwells (Costar, Cambridge, Mass.) of a pore size of 8 Mm were coated with 100 μL of 100 ng/mL human fibronectin (Sigma) for 2 h at room temperature. After removal of the fibronectin, 600 μL of RPMI with 5% BSA in the presence of histamine (ranging from 1.25-20 μM) was added to the bottom chamber. To test the various histamine receptor antagonists, 10 μM solutions of the compounds were added to the top and bottom chambers. Mast cells (2×10⁵/well) were added to the top chamber. The plates were incubated for 3 h at 37° C. Transwells were removed and the number of cells in the bottom chamber was counted for 60 sec using a flow cytometer.

Results

Histamine Mediates Chemotaxis Through H4 Receptor

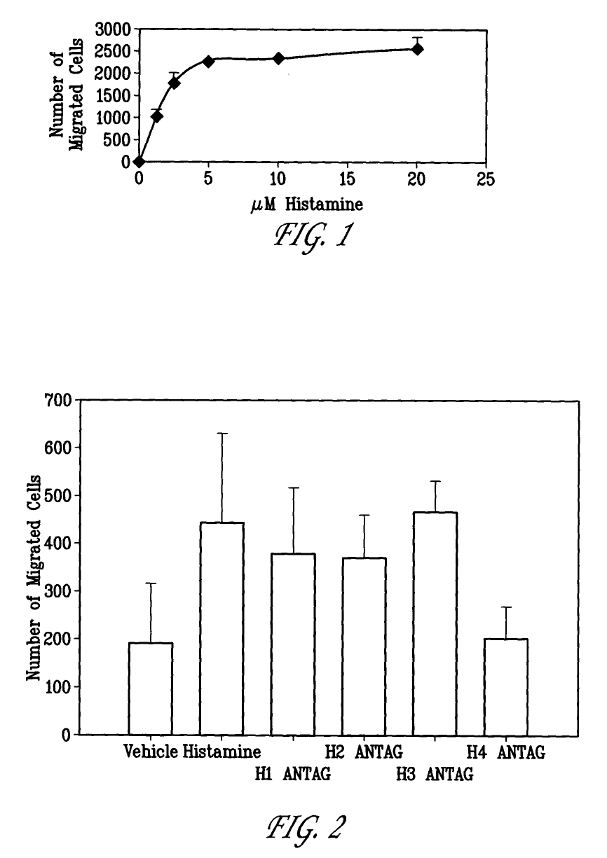

Chemotactic ability of mast cells towards histamine was investigated using a transwell system. Mast cells were added to the upper chamber, while histamine was added to the lower chamber. Histamine induced a dose-dependent increase in migrated mast cells in the lower chamber (FIG. 1).

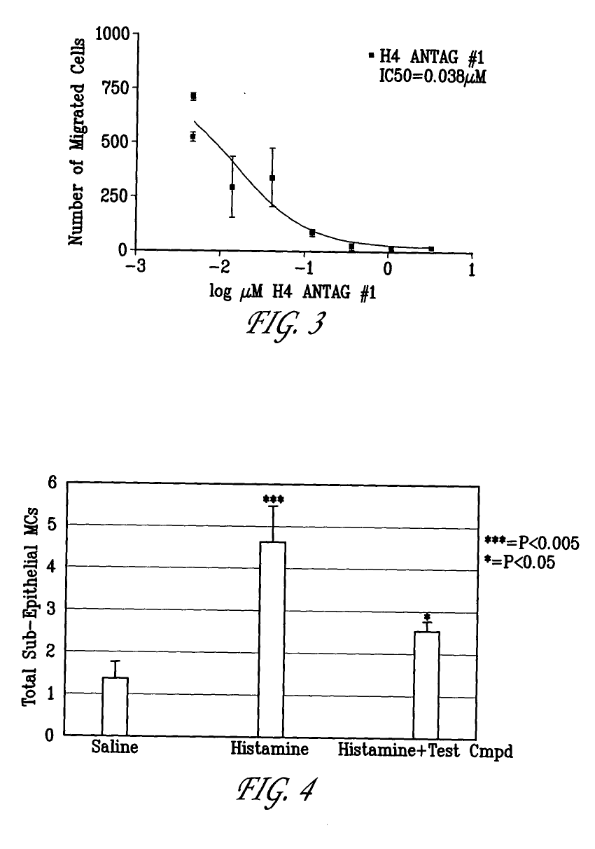

Specific histamine receptor antagonists were used to sort out which histamine receptor is responsible for the chemotaxis towards histamine. Antagonists specific for the histamine H1, H2 or H3 receptors did not alter the histamine-induced chemotaxis (FIG. 2). However, a specific histamine H4 receptor antagonist inhibited mast cell chemotaxis (FIGS. 2 and 3) in a dose-dependent manner.

The results set forth in Table 4 below show a positive correlation between the Kᵢ of the compound and its ability to inhibit mast cell chemotaxis; i.e., in general, the more potent the compound the better the inhibition.

TABLE 4

| | Kᵢ | % Inh | % Inh |
|---|---|---|---|
| COMPOUND | (nM) | 10 μM | 1 μM |
| (5-Chloro-7-methyl-1H-indol-2-yl)-(4-methyl-piperazin-1-yl)-methanone | 1 | 97 | 100 |
| (7-Amino-1H-indol-2-yl)-(4-methyl-piperazin-1-yl)-methanone | 7 | | 99 |
| (5-Chloro-1H-indol-2-yl)-piperazin-1-yl-methanone | 10 | | 99 |
| (5,7-Difluoro-1H-indol-2-yl)-(4-methyl-piperazin-1-yl)-methanone | 19 | | 100 |
| (4-Methyl-piperazin-1-yl)-(3-methyl-4H-thieno[3,2-b]pyrrol-5-yl)-methanone | 21 | | 60 |
| (2-Chloro-6H-thieno[2,3-b]pyrrol-5-yl)-(4-methyl-piperazin-1-yl)-methanone | 25 | 106 | 103 |
| (5-Chloro-1H-benzoimidazol-2-yl)-(4-methyl-piperazin-1-yl)-methanone | 25 | 97 | 84 |
| (5,6-Difluoro-1H-benzoimidazol-2-yl)-(4-methyl-piperazin-1-yl)-methanone | 28 | 97 | 72 |
| (2-Chloro-4H-thieno[3,2-b]pyrrol-5-yl)-(4-methyl-piperazin-1-yl)-methanone | 40 | | 92 |
| (5-Chloro-1H-benzoimidazol-2-yl)-piperazin-1-yl-methanone | 87 | 101 | 8 |
| (2-Chloro-6H-thieno[2,3-b]pyrrol-5-yl)-(hexahydro-pyrrolo[1,2-a]pyrazin-2- | 176 | 0 | 0 |
| yl)-methanone | | | |
| 5-Methyl-1H-benzoimidazole-2-carboxylic acid (8-methyl-8-aza- | 613 | 27 | 66 |
| bicyclo[3.2.1]oct-3-yl)-amide | | | |
| (3-Bromo-4H-thieno[3,2-b]pyrrol-5-yl)-(3-methyl-piperazin-1-yl)-methanone | 980 | 0 | 0 |
| Indole control | >10K | 0 | 0 |
| Bicyclic pyrrole control | >10k | 0 | 0 |
| Benzoimidazole control | >10k | 0 | 0 |

### EXAMPLE 5

### The Inhibition of Histamine-Induced Mast Cell Chemotaxis by Histamine H4 Receptor Antagonists in vivo

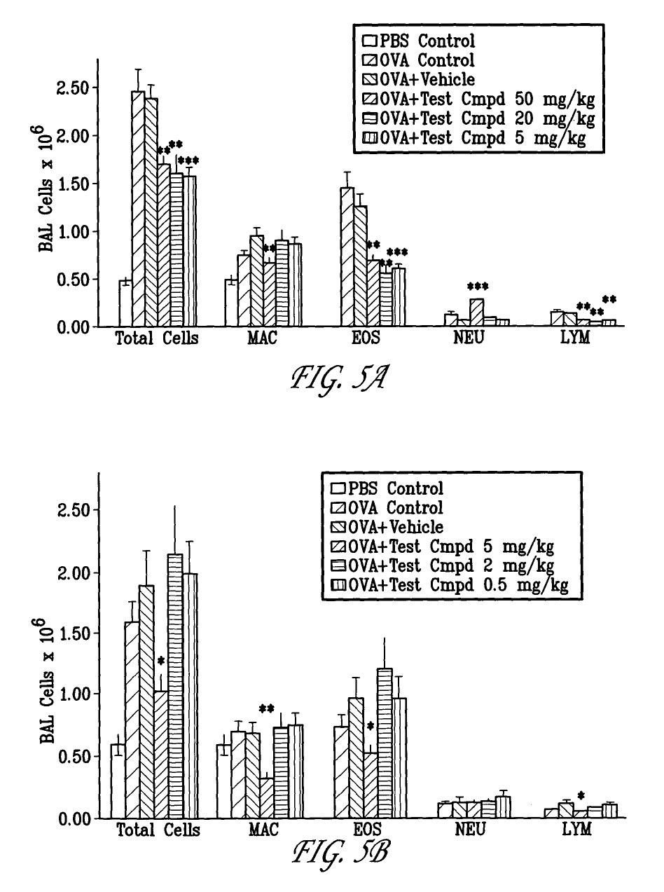

Groups of ten female Balb/c mice (8-12 weeks) were exposed to an aerosol of saline control or 0.1 M histamine for 20 min on 2 consecutive days. Histamine-aerosolized mice were pre-dosed 15 min prior to each aerosol with either saline or 20 mg/kg of the H4 modulator (5-chloro-1H-indol-2-yl)-(4-methyl-piperazin-1-yl)-methanone via the subcutaneous route (5 ml/kg). Four hours after the final aerosol administration, mice were sacrificed through pentobarbitone overdose and a section of the trachea was removed and fixed in formalin. Paraffin embedding and longitudinal sectioning of tracheas was performed and the mast cells were stained with toluidine blue. Mast cells were quantified as sub-mucosal or sub-epithelial depending on their location within each tracheal section. Statistics were performed using Students unpaired t-test. The results (FIG. 4) show that histamine induces a migration of mast cells into the sub-epithelial space. Such movement is similar to what is thought to occur upon allergen exposure. This migration can be blocked by a specific H4 receptor antagonist.

### EXAMPLE 6

### Inhibition of Mast Cell Chemotaxis by H₄ Receptor Antagonist in an Animal Model of Asthma and Allergic Rhinitis

The animal model set forth in this example is used to test the observation that mast cells accumulate in response to allergic inflammation and that this accumulation can be blocked by H4 receptor antagonists. Compounds of the present invention are tested in this model to demonstrate their use as treatments for allergic rhinitis or asthma. Mice are sensitized by intraperitoneal injection of ovalbumin/Alum (10 μg in 0.2 mL Al(OH)₃; 2%) on Day 0 and Day 14. On Day 21 through 23, mice are challenged by PBS or ovalbumin, and sacrificed 24 h after the last challenge on Day 24. A section of the trachea is removed and fixed in formalin. Paraffin embedding and longitudinal sectioning of tracheas are performed, followed by staining of mast cells with toluidine blue. Alternatively, tracheas are frozen in OCT for frozen sectioning, and mast cells are identified by IgE staining. Mast cells are quantified as sub-mucosal or sub-epithelial depending on their location within each tracheal section. Exposure to allergen increases the number of sub-epithelial mast cells, and the ability of H4 receptor antagonists to block this effect is measured.

### EXAMPLE 7

### The Inhibition of Basophil Chemotaxis by Histamine H4 Receptor Antagonists

Basophils are isolated from human blood using standard methods (Tsang et al., Immunological Methods (2000) 233(1-2):13-20). The chemotaxis assays can be carried out using transwells (Costar, Cambridge, Mass.) of a pore size of 8 μm coated with 100 μL of 100 ng/mL human fibronectin (Sigma) for 2 h at room temperature. After removal of the fibronectin, 600 μL of RPMI with 5% BSA in the presence of histamine (ranging from 1.25-20 μM) is added to the bottom chamber. To test the various histamine receptor antagonists, 10 μM solutions of the compounds can be added to the top and bottom chambers. Basophils can be added to the top chamber. The plates are incubated for 3 h at 37° C. Transwells are removed and the number of cells in the bottom chamber can be counted for 60 sec using a flow cytometer or can be quantified by staining using Wright's staining.

### EXAMPLE 8

### Measurement of Increases in Mast Cell and Basophil Populations in Patients After Exposure to Antigen

Patients with allergies to particular allergens such as cat dander or grass pollens are challenged with the appropriate antigens via direct bronchial administration or other methods. The number of mast cells and basophils in the bronchial mucosa and nasal mucosa are quantified using standard immunohistochemical staining methods after tissue biopsy. The effects of a histamine H4 receptor modulator on the increase in mast cells and basophils after antigen challenge are studied by administrating the modulator by a number of different routes before the antigen challenge.

### EXAMPLE 9

### Measurement of Tissue Mast Cell and Basophil Populations in Patients

Patients with allergic rhinitis and/or asthma are known to have increases in both mast cells and basophils in their airways compared to healthy subjects. The effects of a histamine H4 receptor modulator on the population of mast cells and basophils resident in the airways is examined by administering the modulator by a number of different routes for a given period of time. A comparison of the number of mast cells and basophils found in the airways can be made before, during and after treatment. The cells are quantified using standard immunohisto-chemical staining methods after tissue biopsy.

### EXAMPLE 10

### The Treatment of Asthma or Allergic Responses in Patients Using Histamine H4 Receptor Antagonists

Allergic or asthmatic patients are given a histamine H4 receptor antagonist or placebo for a given period of time. Throughout the course of the treatment asthma, the patient's severity score, forced expiratory volume in one second (FEV1), and bronchial hyperreactivity (BHR) for bronchocontrictors is measured. Decreases in the asthma severity score, increases in FEV1 and decreases in BHR after treatment with a histamine H4 receptor antagonist are all indicative of a positive effect on the disease. In addition, decreases in the inflammatory response upon treatment with histamine H4 receptor antagonists may be determined by changes in serum concentrations of soluble interleukin 2 receptor (sIL-2R), IL-4, and soluble intercellular adhesion molecule 1 (sICAM-1); peripheral blood eosinophil count; and eosinophilic cationic protein (ECP). In addition, bronchial biopsies may be used to quantitate the change in inflammatory cell populations, such as eosinophils, T cells, mast cells, basophils, and the like.

### EXAMPLE 11

### The Treatment of Allergic Responses in Patients Using Histamine H4 Receptor Antagonists

Patients with allergic rhinitis are given a histamine H4 receptor antagonist or placebo for a given period of time. Efficacy is measured by comparing daytime nasal symptoms score (average of congestion, itching, and sneezing), eye symptoms, nighttime symptoms, individual daytime nasal symptoms, global evaluations (patient's and physician's), and quality-of-life scores. In addition, the effects on allergic conjunctivitis may be quantified using measurements of ocular redness, itching, and days without symptoms.

### EXAMPLE 12

### The Inhibition of Histamine-Induced Eosinophil Shape Change by Histamine H4 Receptor Antagonists

This example demonstrates that histamine H4 receptor antagonists can block the shape change response of human eosinophils to histamine. Shape change in eosinophis is an early event that leads to one or more of several different biological outcomes, including adhesion, chemotaxis, degranulation and phagocytosis. These downstream events are associated with allergic and other inflammatory/immune responses, including allergic rhinitis and asthma.

Methods

Human granulocytes were isolated from human blood by a Ficoll gradient. The red blood cells were lysed with 5-10× Qiagen lysis buffer at room temperature for 5-7 min. Granulocytes were harvested and washed once with FACS buffer. The cells were resuspended at a density of 2×10⁶ cells/mL in reaction buffer. To test inhibition by specific histamine receptor antagonists, 90 μL of the cell suspension (˜2×10⁵ cells) was incubated with 10 μM of one of the various test compound solutions. After 30 min, 11 μL of one of the various concentrations of histamine was added. Ten minutes later, the cells were transferred to ice and fixed with 250 μL of ice-cold fixative buffer (2% formaldehyde) for 1 min. The shape change was quantitated using a gated autofluoescence forward scatter assay (GAFS) (Byran et al., Am. J Crit. Care Med. 165:1602-1609, 2002).

Results

The data in the following table show that histamine induces a dose-dependent shape change in eosinophils. Histamine receptor (HR) antagonists were used to sort out which histamine receptor is responsible for the shape change. Antagonists specific for the histamine H1 receptor (diphenhydramine) or the H2 receptor (ranatidine) did not alter the histamine-induced shape change. However, a dual H3/H4 antagonist (thioperamide) and a specific histamine H4 receptor antagonist, (5-chloro-1H-indol-2-yl)-(4-methyl-piperazin-1-yl)-methanone (Kᵢ=5 nM), inhibited histamine-induced eosinophil shape change with IC₅₀'s of 1.5 and 0.27 μM, respectively. The data are presented in Table 5.

TABLE 5

| Histamine | Fold Change | | | | |
|---|---|---|---|---|---|
| (μM): | 10 | 1 | 0.1 | 0.01 | 0 |
| No HR | 1.34 | 1.31 | 1.21 | 1.01 | 1.00 |
| Antagonist | | | | | |
| 10 μM H₄ | 1.09 | 1.05 | 1.05 | 1.01 | 1.00 |
| Antagonist | | | | | |
| 10 μM | 1.08 | 1.05 | 1.01 | 1.04 | 1.00 |
| Thiop | | | | | |
| 10 μM | 1.63 | 1.50 | 1.18 | 1.03 | 1.00 |
| Diphen | | | | | |
| 10 μM | 1.64 | 1.49 | 1.21 | 1.04 | 1.00 |
| Ranat | | | | | |

### EXAMPLE 13

### H4 Receptor Antagonists Modulate Response in a Model of Human Allergic Inflammation

This example demonstrates that H4 receptor antagonists modulate allergic response in animals to ovalbumin-induced lung inflammation, a common animal model for human allergic inflammation.

Methods

Sensitizing and Challenging Animals

Mice (n=8 per group) were sensitized by intraperitoneal injection of ovalbumin/alum on Days 0 and 14. On each of Days 21 through 24, mice were pretreated for 15 min with vehicle or with (5-chloro-1H-benzoimidazol-2-yl)-(4-methyl-piperazin-1-yl)-methanone prior to a 20 min challenge with PBS or ovalbumin.

Administration of Exemplary H4 Receptor Antagonists

In one experiment, (5-chloro-1H-benzoimidazol-2-yl)-(4-methyl-piperazin-1-yl)-methanone was administered p.o. at 5, 20, or 50 mg/kg. In a second experiment, (5-chloro-1H-benzoimidazol-2-yl)-(4-methyl-piperazin-1-yl)-methanone was administered p.o. at 0.5, 2, or 5 mg/kg. In a third experiment, (5-chloro-1H-indol-2-yl)-(4-methyl-piperazin-1-yl)-methanone was administered s.c. at 20, 60, or 100 mg/kg.

Cell Measurement/Data Collection

Twenty-four hours after the last challenge, mice were sacrificed and the total number of cells, as well as a differential cell count in the bronchavaeolar lavage (BAL) fluid, was determined.

Airway Hyper-Responsiveness Monitoring

Airway hyper-responsiveness in ovalbumin-sensitized animals was monitored in conscious mice by whole body plethysmography (Buxco). Mice (n=8 per group) were treated as described above for the cell measurements.

Results

Analysis of BAL Fluid

After challenge with ovalbumin there was a dramatic increase in the total number of cells in the BAL fluid that was mainly due to increases in the number of eosinophils. As shown in FIG. 5, both the total cell number and the number of eosinophils were significantly reduced at all doses of 5 mg/kg and greater of (5-chloro-1H-benzoimidazol-2-yl)-(4-methyl-piperazin-1-yl)-methanone. The maximum reduction in the number of eosinophils was 50%. The same doses which provided maximum reduction in eosinophils significantly reduced the number of lymphocytes as well. At the highest dose tested (50 mg/kg), there was a reduction in the number of macrophages and an increase in the number of neutrophils. A reduction in macrophages was also seen at a dose of 5 mg/kg in the second experiment. These data indicate that H4 receptor antagonists such as (5-chloro-1H-benzoimidazol-2-yl)-(4-methyl-piperazin-1-yl)-methanone have anti-inflammatory properties.

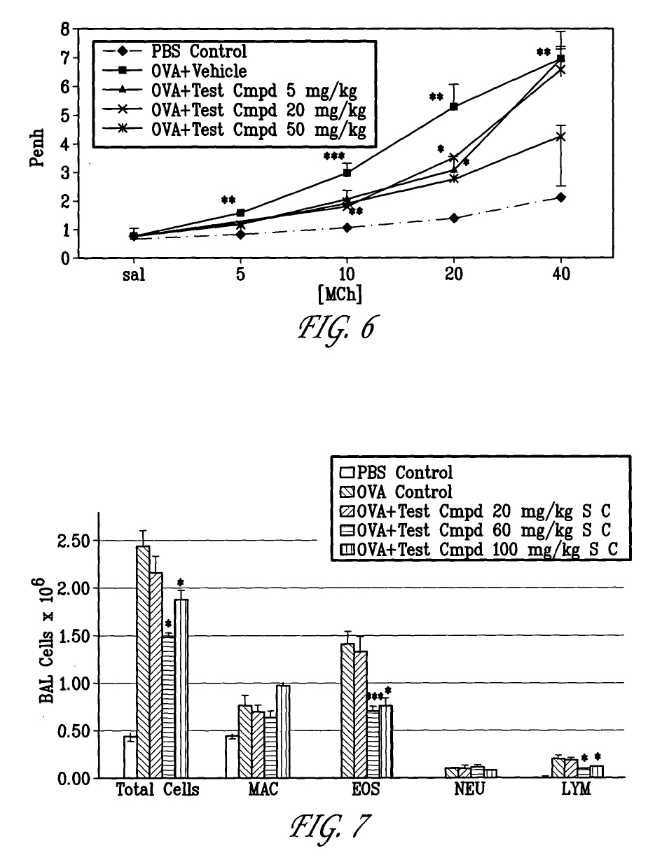

Another H4 receptor antagonist, (5-chloro-1H-indol-2-yl)-(4-methyl-piperazin-1-yl)-methanone, was also tested in this model. FIG. 6 shows the results for the differential cell count in the BAL fluid. Both the total cell number and the number of eosinophils were significantly reduced at 60 and 100 mg/kg of (5-chloro-1H-indol-2-yl)-(4-methyl-piperazin-1-yl)-methanone. The maximum reduction in the number of eosinophils was 50%. At the same doses that provided reduction in eosinophils, the number of lymphocytes was significantly reduced.

Analysis of Airway Hyper-Responsiveness

The response of airway hyper-responsiveness to methacholine is shown in FIG. 7. Twenty-four hours after the final ovalbumin challenge of sensitized animals, there was a dramatic increase in the response of the airway to methacholine as measured by Penh (enhanced pause), an index of bronchoconstriction. This hyper-responsiveness was reduced by treatment with (5-chloro-1H-benzoimidazol-2-yl)-(4-methyl-piperazin-1-yl)-methanone at any of the first three doses. These data indicate that H4 receptor antagonists not only block airway inflammation but also affect allergic airway hyperactivity, and thus, will be useful in treating allergies and/or asthma in humans.

The features and advantages of the invention are apparent to one of ordinary skill in the art. Based on this disclosure, including the summary, detailed description, background, examples, drawings and claims, one of ordinary skill in the art will be able to make modifications and adaptations to various conditions and usages. These other embodiments are also within the scope of the invention. Publications referenced herein are incorporated in their entireties by such reference.

What is claimed:

1. A method of identifying compounds that modulate mammalian histamine H4 receptor activity, comprising:
a) combining a putative modulator compound of mammalian histamine H4 receptor activity with mammalian histamine H4 receptor and a known histamine receptor H4 ligand; and
b) measuring an effect of the modulator on the protein function or its ability to bind the ligand, wherein said effect is a modulation selected from the group consisting of inhibition, activation, antagonist, agonist, and inverse agonist activity,
wherein said modulator compound is a modulator of mast cell chemotaxis or basophil chemotaxis.

2. The method of claim 1, wherein the effect measured in step b) is inhibition by competition between the modulator of step a) and a known histamine receptor H4 ligand for binding to the receptor.

3. The method of claim 1, wherein the effect measured in step b) is modulation of a histamine H4 receptor intracellular second messenger.

4. The method of claim 3, wherein the intracellular second messenger is selected from the group consisting of cAMP, calcium, and a reporter gene product.

5. The method of claim 1, wherein said compound is a modulator of mast cell chemotaxis.

6. The method of claim 1, wherein said compound is a modulator of basophil chemotaxis.

7. A compound identified using the method of claim 1, wherein said compound is an inhibitor of a mammalian histamine H4 receptor function and an inhibitor of mast cell chemotaxis or basophil chemotaxis in vivo or in vitro.

8. A compound identified using the method of claim 1, wherein said compound is an agonist, antagonist, or inverse agonist of a mammalian histamine H4 receptor.

9. A compound identified using the method of claim 1, wherein said compound modulates expression of a gene encoding the mammalian histamine H4 receptor.

10. A monospecific antibody immunologically reactive with a mammalian histamine H4 receptor protein, wherein said antibody modulates mast cell chemotaxis or basophil chemotaxis.

11. The antibody of claim 10, wherein the antibody blocks histamine binding or activation of the mammalian histamine H4 receptor protein.

12. A pharmaceutical composition comprising a compound active in the method of claim 1 and a pharmaceutically acceptable carrier wherein said compound is a modulator of asthma or allergic responses.

13. A method of treating a patient in need of such treatment to modulate asthma or allergic responses or a disease or condition that is mediated by asthma or allergic responses and histamine H4 receptor comprising administration of the pharmaceutical composition of claim 12.

14. A pharmaceutical composition comprising a compound active in the method of claim 1 and a pharmaceutically acceptable carrier wherein said compound is a modulator of mast cell chemotaxis.

15. A method of treating a patient in need of such treatment to modulate asthma or allergic responses or a disease or condition that is mediated by mast cell chemotaxis and histamine H4 receptor comprising administration of the pharmaceutical composition of claim 14.

16. A pharmaceutical composition comprising a compound active in the method of claim 1 and a pharmaceutically acceptable carrier wherein said compound is a modulator of basophil chemotaxis.

17. A method of treating a patient in need of such treatment to modulate asthma or allergic responses or a disease or condition that is mediated by basophil chemotaxis and histamine H4 receptor comprising administration of the pharmaceutical composition of claim 16.

18. A pharmaceutical composition comprising a compound that modulates mammalian histamine H4 receptor activity and a pharmaceutically acceptable carrier, wherein said compound is a modulator of mast cell or basophil chemotaxis in vitro or in vivo.

19. The pharmaceutical composition of claim 18, wherein the compound inhibits mast cell chemotaxis.

20. The pharmaceutical composition of claim 18, wherein the compound inhibits basophil chemotaxis.

21. The pharmaceutical composition of claim 18, wherein the compound is an agonist, antagonist, or inverse agonist of a mammalian histamine H4 receptor.

22. The pharmaceutical composition of claim 18, wherein the compound modulates expression of a gene encoding the mammalian histamine H4 receptor.

23. The pharmaceutical composition of claim 18, wherein the compound further modulates eosinophil shape change in vitro or in vivo.

24. A method of treating a patient in need of such treatment to modulate asthma or allergic responses or a disease or condition that is mediated by mast cell or basophil chemotaxis and histamine H4 receptor comprising administering a pharmaceutical composition comprising a compound that modulates mammalian histamine H4 receptor activity and a pharmaceutically acceptable carrier, wherein said compound is a modulator of mast cell or basophil chemotaxis in vitro or in vivo.

25. The method of claim 24, wherein the pharmaceutical composition comprises a compound that inhibits mast cell chemotaxis.

26. The method of claim 24, wherein the pharmaceutical composition comprises a compound that inhibits basophil chemotaxis.

27. The method of claim 24, wherein the pharmaceutical composition comprises a compound that is an agonist, antagonist, or inverse agonist of a mammalian histamine H4 receptor.

28. The method of claim 24, wherein the pharmaceutical composition comprises a compound that modulates expression of a gene encoding the mammalian histamine H4 receptor.

29. The method of claim 24, wherein the pharmaceutical composition comprises a compound that modulates eosinophil shape change in vitro or in vivo.

30. A method of identifying compounds that modulate mammalian histamine H4 receptor-mediated chemotaxis of mast cells to histamine, the method comprising:
a) in the presence or absence of a test compound being tested as a histamine H4 receptor modulator, placing mast cells in proximity to histamine under conditions enabling movement of the mast cells toward the histamine; and
b) measuring an effect of the histamine H4 receptor modulator on the movement of the mast cells toward the histamine, wherein an increase or decrease in rate of mast cell movement toward the histamine or in number of mast cells that move toward the histamine is indicative that the test compound modulates histamine H4 receptor-mediated chemotaxis of the mast cells to histamine.

31. A method of determining if a histamine H4 receptor modulator modulates sub-epithelial accumulation of mast cells in a mammalian trachea in response to exposure to histamine or an allergen, the method comprising:
a) in the presence or absence of pre-treatment with a histamine H4 receptor modulator, exposing a mammal to an aerosol comprising histamine or an allergen under a regimen that would, in the absence of the modulator, result in a pre-determined amount of sub-epithelial mast cell accumulation in the mammal's trachea; and
b) comparing sub-epithelial mast cell accumulation in the mammal's trachea in the presence and absence of the histamine H4 receptor modulator, a change in the sub-epithelial mast cell accumulation in the presence of the modulator as compared to in the absence of the modulator being indicative that the histamine H4 receptor modulator modulates the sub-epithelial accumulation of mast cells in the mammal's trachea in response to the exposure to histamine or an allergen.
